(19) United States
(12) Patent Application Publication
(10) Pub. No.: US 2002/0193831 A1
(43) Pub. Date: Dec. 19, 2002

(54) Method and apparatus for the treatment of cosmetic skin conditions

(76) Inventor: Edward Dewey Smith III, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/132,026

(22) Filed: Apr. 25, 2002

Related U.S. Application Data
(60) Provisional application No. 60/286,844, filed on Apr. 26, 2001.

(51) Int. Cl. A61N 1/18

(52) U.S. Cl. 607/2

(57) ABSTRACT

The present invention relates to a method for the treatment of cosmetic skin conditions such as regional fat deposits, and in particular for the treatment of cellulite using an electrostatically discharged current. The method comprises treating a selected area of a cosmetic skin condition by the electrostatic application, from a storage medium, of a current between the storage medium and the selected area. The invention is further concerned with a kit for treating a selected area of cosmetic skin conditions such as regional fat deposits, and in particular for the treatment of cellulite. The kit includes a device comprising an electrostatic storage medium from which, in use, a current is electrostatically discharged between the device and the selected area and a composition adapted to be applied on or adjacent the selected area. The kit preferably further includes a device comprising at least one alternative energy form selected from the group comprising light, ultrasound, active massage, heat, compression, static magnets and combinations thereof.

### CROSS REFERENCE TO RELATED APPLICATIONS

This application claims the benefit of U.S. Provisional Application No. 60/286,844, filed Apr. 26, 2001.

### FIELD OF THE INVENTION

The present invention relates to a method for the treatment of cosmetic skin conditions such as regional fat deposits, and in particular for the treatment of cellulite using an electrostatically discharged current. The method comprises treating a selected area of a cosmetic skin condition by the electrostatic application, from a storage medium, of a current between the storage medium and the selected area.

The invention is further concerned with a kit for treating a selected area of cosmetic skin conditions such as regional fat deposits, and in particular for the treatment of cellulite. The kit includes a device comprising an electrostatic storage medium from which, in use, a current is electrostatically discharged between the device and the selected area and a composition adapted to be applied on or adjacent the selected area. The kit preferably further includes a device comprising at least one alternative energy form selected from the group comprising light, ultrasound, active massage, heat, compression, static magnets and combinations thereof.

### BACKGROUND OF THE INVENTION

As we age, and as a normal course of hormonal fluctuations, environmental influences and individual genetic tendencies, skin elasticity is gradually reduced. At the same time, lean tissue mass decreases and adipose tissue increases. Generally speaking, adipose tissue tends to concentrate the body's fat stores in a few regional sites of the body, such as the mid-section, the thighs and buttocks, and/or the back of the arms. In some regions, especially the legs, bulging of fat chambers near the skin's surface can cause dimpling of the skin at the attachment points of the skin's underlying structural fibrous strands. This regional fat deposit is termed cellulite and it occurs most often on the thighs, hips, waist, buttocks and upper arms of women. Cellulite is a cosmetic rather than a medical condition.

The dimpling of the skin affected by cellulite is also known as the “orange peel” effect, and it is an undesirable cosmetic condition that affects women of all ages and sizes, although it is generally more prevalent in women who are overweight to some degree. In a society that is increasingly concerned with image, women have resorted to many methods to try to rid themselves of cellulite.

To date, many creams for the treatment of cellulite have been available on the market. Relatively expensive to buy, their results are often minimal and short-lived. Dry-brushing is another method suggested for the treatment of cellulite, which involves the frequent brushing of oneself with relative vigour with the bristles of a suitable brush. The method of dry-brushing, however, leaves the skin feeling relatively uncomfortable and raw, and it also often has a minimal effect on the cellulite.

There has been a desire in recent years to provide a different method of treating regional fat deposits including cellulite which is both effective and relatively pain free.

Cellulite is not the only cosmetic condition that concerns women. Stretch marks are another example of a cosmetic condition which affects not only women, but also men. Stretch marks can form at various stages of a person's life, for example, at puberty, during pregnancy in the case of women, or generally when a person gains a substantial amount of weight. Stretch marks are most commonly found on the thighs, buttocks and abdomen, but also quite frequently appear on other areas, the upper arms for example. The stretch marks appear as generally purple blemishes on the skin, generally quite long and thin, with a length dependable on the position of the body on which they are found and the reason for the formation of the stretch marks. Over time, the stretch marks fade in colour and eventually have a silver appearance. It is virtually impossible to rid oneself of stretch marks using conventional methods. Loss of weight will result in their appearance being less noticeable but they are still present on the skin. Creams are available on the market which claim to reduce the appearance of the stretch marks, but the effect of these creams are generally minimal and short-lived, similar to the effects of the creams for the treatment of cellulite.

The application of electrostatic fields in various medical treatments is well known, and in particular is utilised in the acceleration of the healing of wounds, and in the transdermal delivery of drugs to a patient.

EP 51087 discloses a method and apparatus for the internal release of a pharmaceutical product within the body of a patient. The apparatus comprises a support which, in use, is implanted within the patient's body, and which is connected to a power source arranged to establish an electric field about the support. The apparatus further includes a supply of the pharmaceutical product disposed about the support such as to be driven outwardly into the patient's body under the influence of the electric field.

EP 254166 concerns a method and apparatus for the dielectrophoretic delivery of a drug into the systemic circulation system of a patient. The method and apparatus utilise a reservoir of a colloidal drug which, in use, contacts the patient's skin on one side thereof. Located in contact with the opposed side of the reservoir, distal the patient's skin, is an electric field generating device, which may be in the form of an electret. The electric field generated passes through the reservoir and the patient's skin, causing particles of the drug to become polarised and therefore migrate through the skin by means of dielectrophoresis.

U.S. Pat. No. 4,142,521 discloses the use of an electric field in the accelerated healing of wounds. The apparatus disclosed consists of a bandage contained within which is an electret, thereby establishing an electric field which emanates from the bandage. In use, the bandage is adhered to the skin of the patient adjacent the site of the wound, thereby exposing the wound to the electric field.

WO 97/04830 concerns a method of increasing the efficacy of a bioactive agent which has been administered to a patient, by means of the application of a polarising field to either a portion of, or the entire patient's body. The application of the field to the body affects the natural charge distribution within the bodies cells, such as to increase the effectiveness of the agent by improving the receptivity or susceptibility of said cells to the agent. The polarising field may be either electric or magnetic.

WO 00/17322 discloses a method for stimulating one or more biological activities within cells, by means of applying electromagnet stimulation to said cells. This is achieved by contacting the particular cells with an electroactive material, preferably an electroactive polymer, to which is already attached mammalian tissue, and applying electromagnetic radiation to the electroactive material. The method is particularly concerned with the regeneration of bone cells.

EP 788811 discloses a device for carrying out endermic electrotherapeutic treatments which comprises a pulsed current generator and a garment to be worn by a patient, in which the garment is comprised of a plurality of electrically conductive discrete portions for contact with the patients skin and connected to insulating portions intermediate between said conductive portions, the conductive portions being provided with respective terminals connected to the current generator by a conductor. The insulating portions are provided intermediate said conductive portions in order to define pairs of positive and negative electrodes through which current is actively passed. The device is primarily intended for use in the treatment of cellulite. However, EP 788811 neither discloses nor suggests the electrostatic application of current to a selected area of a cosmetic skin condition such as regional fat deposits, and in particular cellulite.

The electrostatic application of current to a selected area requires only a single contact surface, as opposed to one or more pairs of electrodes as disclosed in the known art, the electrostatic application further ensuring the discharge of a microcurrent which is surprisingly found to be particularly beneficial in the treatment of a selected area of a cosmetic skin condition such as regional fat deposits, and in particular cellulite.

It is an object of the present invention to provide a method which enables the effective treatment of a selected area of the skin and/or subcutaneous tissue such as regional fat deposits, and in particular the treating of a selected area of a cosmetic skin condition by the electrostatic application, from a storage medium, of a current between the storage medium and the selected area.

It is a further object of the present invention to provide a kit for the effective treatment of an area of the skin and/or subcutaneous tissue, and in particular for the treatment of cosmetic skin conditions such as regional fat deposits, including cellulite in a selected area, by the electrostatic application, from a storage medium, of a current between the storage medium and the selected area. It is a still further object of the present invention to provide a method or kit which uses the electrostatic application, from a storage medium, of a current between the storage medium and the selected area which are suitable for domestic use or unsupervised use in a clinic. These, in addition to other objects of the invention, will be readily apparent from the following description.

### SUMMARY OF THE INVENTION

The present invention relates to a method for the treatment of a selected area of the skin and/or subcutaneous tissue, and in particular for the treatment of a selected area of a cosmetic skin condition such as regional fat deposits, including cellulite, comprising the electrostatic application, from a storage medium, of a current between the storage medium and the selected area.

The present invention also relates to a method for the treatment of a selected area of the skin and/or subcutaneous tissue, and in particular for the treatment of a selected area of a cosmetic skin condition such as regional fat deposits, including cellulite, comprising the steps of exposing the selected area to the electrostatic application, from a storage medium, of a current between the storage medium and the selected area; and exposing the selected area to a source of at least one alternative energy form selected from the group comprising light, ultrasound, active massage, heat, compression, static magnets and combinations thereof. Of the alternative energy forms, the use of ultrasound is the least preferred.

The present invention further relates to a kit for the treatment of skin and/or subcutaneous tissue, and in particular for the treating of a selected area of a cosmetic skin condition such as regional fat deposits, including cellulite, the kit including a device comprising an electrostatic storage medium from which, in use, a current is electrostatically discharged between the device and the selected area; and a composition adapted to be applied on or adjacent the selected area.

The present invention additionally relates to a kit for the treatment of skin and/or subcutaneous tissue, and in particular for the treating of a selected area of a cosmetic skin condition such as cellulite, the kit including a device comprising an electrostatic storage medium from which, in use, a current is electrostatically discharged between the device and the selected area; a topical composition adapted to be applied on or adjacent the selected area; a device comprising at least one alternative energy form selected from the group comprising light, ultrasound, active massage, heat, compression, static magnets, and combinations thereto; and an oral composition or combinations thereof.

### BRIEF DESCRIPTION OF FIGURES

FIG. 1 illustrates a schematic diagram of a device according to the present invention;

FIG. 2 illustrates the steady state current flow, through the device of FIG. 1, for one half of an alternating current cycle; and

FIG. 3 illustrates the steady state current flow, in the device of FIG. 1, for the second half of the alternating current cycle of FIG. 2.

### DETAILED DESCRIPTION OF THE INVENTION

All publications cited herein are hereby incorporated by reference in their entirety, unless otherwise indicated.

As used herein, the term “subcutaneous tissue” means tissue lying beneath the skin and includes adipose tissue and subcutaneous fat.

As used herein, the term “regional fat deposits” means deposits of fat, which generally do not respond to dieting and exercise, and is intended to embrace cellulite as a subset thereof.

As used herein, the term “electrostatic” means pertaining to static electricity or an electric charge at rest—electrostatic charge is that charge stored in a capacitor or on the surface of an insulating material.

As used herein, the term “electret” means a permanently or semi-permanently polarized piece of dielectric material produced by heating the material and placing it in a strong electric field during cooling. The electric field of an electret corresponds somewhat to the magnetic field of a permanent magnet.

As used herein, the term “ultrasound” means pressure waves having a frequency of at least 16 kHz, preferably at least 20 kHz, the application of which may be either continuous or pulsed. Pulsed ultrasound is effectively a train of pulses. For example, ultrasound can be delivered in an “on-off” mode, where the unit pulses on for 0.2 seconds, then off for 0.8 seconds, with this cycle being repeated indefinitely. Pulsing is typically used for high energy input uses. The “off” time allows heat that may have built up in one area to diffuse away, such that no localised hot spots result. For the present invention, pulsing is acceptable and will produce the desired results, but continuous wave ultrasound is preferred.

As used herein, the term “compression” means the application of static pressure by wrapping or otherwise, increasing the pressure in the tissues.

As used herein, the term “static magnet” means a magnet with a static magnetic field having an intensity of from about 100 to about 2000 gauss, the magnet in use, imparting a bipolar or alternating magnetic polarity to the body of a user. The use of static magnets in the treatment of regional fat deposits such as cellulite preferably involves exposing an area of skin and/or subcutaneous tissue, such as regional fat deposits and in particular cellulite in human skin, to the static magnetic field thereof.

As used herein, the term “light” means monochromatic, dichromatic or multichromatic electromagnetic radiation in the visible or infrared ranges. The use of light in the treatment of cosmetic skin conditions and/or subcutaneous tissue, such as regional fat deposits, and in particular cellulite in human skin, comprises exposing the area of treatment to a source of electromagnetic radiation, preferably having a wavelength of from approximately 600 nanometers to approximately 1100 nanometers. The electromagnetic radiation may be applied by means of one or more LED's, one or more lasers, one or more light bulbs, or any other suitable source of electromagnetic radiation. The electromagnetic radiation may be coherent or non-coherent, pulsed or continuous, or combinations thereof.

As used herein, the term “active massage” means the stimulation of biological tissue by physical or mechanical means. Massaging tissue involves application of stress from outside the tissue, either compression or tension (both are beneficial). The stress can be applied randomly or directionally, for example directed in the direction of the lymph flow. Non-limiting examples of massaging devices are percussive, roller, pinching and vacuum massagers, and combinations thereof. Massage to regional fat deposits such as cellulite skin has the following benefits:

1. Stimulating flow of lymph

2. Increasing blood flow

3. Stretching the connective tissue fibers

4. Remodelling the dermal interface with the subcutaneous adipose tissue

5. Promoting cellular activity via stress-orientation

As used herein, the term “laser” means light amplification by stimulated emission of radiation.

As used herein the term “compression” means the application of static pressure by wrapping or otherwise increasing the pressure in the tissues.

As used herein, the term “pharmaceutical” means a medicinal drug.

As used herein, the term “topical” means designed for or involving local application and action.

As used herein, the term “wearable device” which includes the term “sleeve”, means a substantially flexible section of material in the form of, for example, a wrap, patch, cuff or a bandage which may be placed on/conform to, or which may be held adjacent a selected area of the body. Such a wrap, patch, cuff or bandage may be formed from a substrate, preferably a disposable substrate. The sleeve may, in addition, be dimensioned and adapted to apply compression. The sleeve in the form of a wrap, patch, cuff or bandage may be held in place by the use of straps or fasteners. For example, one side of the sleeve may be connected to the other side of the sleeve, using buttons, Velcro (Trade Mark) or the like. Alternatively, the sleeve may be adapted to form a shape which is specifically designed to fit on an arm, leg, buttocks, stomach or other selected body part. The sleeve may therefore be in the form of a garment such as a sock, trousers, shorts or the like. The material which forms the sleeve is generally flexible and may also have a degree of elasticity. The flexible nature of the sleeve enables the sleeve to conform to the desired shape, and, for example, to enable the sleeve to be pulled up over the selected area of the body. The optionally elastic nature of the sleeve facilitates the sleeve to fit the selected body part in a suitably tight yet comfortable manner.

Cosmetic Skin Conditions

The term “cosmetic skin conditions”, as used herein, includes signs of skin ageing and regional fat deposits including cellulite. “Signs of skin ageing” include, but are not limited to, all outward visibly and tactilely perceptible manifestations as well as any other macro or micro effects due to skin ageing. Such signs may be induced or caused by intrinsic or extrinsic factors, e.g., chronological ageing and/or environmental damage (e.g., sunlight, UV, smoke, ozone, pollutants, stress, etc.). These signs may result from processes which include, but are not limited to, the development of textural discontinuities such as wrinkles, including both fine superficial wrinkles and coarse deep wrinkles, skin lines, facial frown lines, expression lines, rhytides, dermatoheliosis, photodamage, premature skin ageing, crevices, bumps, pits, large pores (e.g., associated with adnexal structures such as sweat gland ducts, sebaceous glands, or hair follicles), “orange peel” skin appearance, dryness, scaliness, flakiness and/or other forms of skin unevenness or roughness; excess skin oil problems such as over-production of sebum, oiliness, facial shine, foundation breakthrough; abnormal desquamation (or exfoliation) or abnormal epidermal differentiation (e.g., abnormal skin turnover) such as scaliness, flakiness, keratoses, hyperkeratinization; inadequate skin moisturization (or hydration) such as caused by skin barrier damage, environmental dryness; loss of skin elasticity (loss and/or inactivation of functional skin elastin) such as elastosis, sagging (including puffiness in the eye area and jowls), loss of skin firmness, loss of skin tightness, loss of skin recoil from deformation; non-melanin skin discoloration such as undereye circles, blotching (e.g., uneven red coloration due to, e.g., rosacea), sallowness (pale colour), discoloration caused by telangiectasia; melanin-related hyperpigmented (or unevenly pigmented) skin regions; post-inflammatory hyperpigmentation such as that which occurs following an inflammatory event (e.g., an acne lesion, in-grown hair, insect/spider bite or sting, scratch, cut, wound, abrasion, and the like); atrophy such as, but not limited to, that associated with ageing or steroid use; other histological or microscopic alterations in skin components such as ground substance (e.g., hyaluronic acid, glycosaminoglycans, etc.), collagen breakdown and structural alterations or abnormalities (e.g., changes in the stratum corneum, dermis, epidermis, the skin vascular system such as telangiectasia); tissue responses to insult such as itch or pruritus; and alterations to underlying tissues (e.g., subcutaneous fat, cellulite, muscles, trabeculae, septae, and the like), especially those proximate to the skin.

Topical Compositions: Carriers

It is envisaged that topical compositions may perform both pharmaceutical and/or cosmetic functions.

The topical carrier compositions of the present invention can comprise a carrier. The carrier should be “dermatologically acceptable”, which means that the carrier is suitable for topical application to the skin, has good aesthetic properties, is compatible with the remaining components, and will not cause any untoward safety or toxicity concerns. A safe and effective amount of carrier is from about 50% to about 99.99%, preferably from about 80% to about 99.9%, more preferably from about 90% to about 98%, most preferably from about 90% to about 95% of the composition.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps (at room temperature). These emulsions can also be delivered in the form of sprays using either mechanical pump containers or pressurised aerosol containers using conventional propellants. These carriers can also be delivered in the form of a mousse. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following four references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; and U.S. Pat. No. 4,254,105 to Fukuda et al., issued Mar. 3, 1981.

A further discussion of suitable carriers is found in U.S. Pat. No. 5,605,894 to Blank et al, and U.S. Pat. No. 5,681,852 to Bissett, both of which are herein incorporated by reference in their entirety.

Topical Compositions: Skin Actives

The compositions of the present invention may optionally comprise one or more skin actives. By the term “skin active” is meant an agent that promotes the growth of healthy skin tissue by, for example, supporting tissue revascularisation. Non-limiting examples of such skin actives include vitamin B3 compounds such as those described in WO 97/39733, published Oct. 30, 1997, to Oblong et al., herein incorporated by reference in its entirety; hydroxy acids such as salicylic acid; anti-oxidants/radical scavengers such as tocopherol and esters thereof; metal chelators, especially iron chelators; retinoids such as retinol, retinyl palmitate, retinyl acetate, retinyl propionate, and retinal; N-acetyl-L-cysteine and derivatives thereof; hydroxy acids such as glycolic acid; keto acids such as pyruvic acid; benzofuran derivatives; and anti-cellulite agents (e.g., xanthines such as caffeine, theophylline); niacinamide, which promotes healthy cell growth in the dermis; polycyclic compounds such as triterperoids (e.g., betulinic acid); and sterols such as stimasterol. Mixtures of any of the above mentioned skin actives may also be used. A more detailed description of these actives is found in U.S. Pat. No. 5,605,894 to Blank et al (previously incorporated by reference).

Other conventional active ingredients, or mixtures thereof, may also be included. These include exfoliation or desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; depilatory agents (e.g., sulfhydryl compounds); skin lightening agents (e.g., arbutin, kojic acid, hydroquinone, ascorbic acid and derivatives such as ascorbyl phosphate salts, placental extract, and the like); moisturizing agents; anti-microbial agents; anti-androgens; and skin protectants. Ultraviolet absorbing agents, often described as sunscreening agents, can be present in a concentration in the range of between about 1% and about 12% by weight, based on the total weight of composition. Preferably, the UV absorbing agents constitute between about 2% and 8% by weight. More preferably, the UV absorbing agents can be present in the composition in a concentration range of between about 4% and about 6% by weight. Of the ultraviolet absorbing agents suitable for use herein, benzophenone-3, octyl-dimethyl PABA (Padimate O), Parsol MCX, and mixtures thereof are particularly preferred. Also useful in topical compositions of the present invention are sunless tanning agents including dihydroxyacetone, glyceraldehyde, indoles and their derivatives, and the like. These sunless tanning agents can also be used in combination with the sunscreen agents.

An optional skin active of the topical compositions of the present invention is a flavonoid compound—an aromatic compound having two substituted benzene rings connected by a chain of three carbon atoms and an oxygen bridge. Flavonoids are broadly disclosed in U.S. Pat. No. 5,686,082 and U.S. Pat. No. 5,686,367, both of which are herein incorporated by reference. Flavonoids suitable for use in the present invention are flavanones selected from the group consisting of unsubstituted flavanones, mono-substituted flavanones, and mixtures thereof; chalcones selected from the group consisting of unsubstituted chalcones, mono-substituted chalcones, di-substituted chalcones, tri-substituted chalcones, and mixtures thereof; flavones selected from the group consisting of unsubstituted flavones, mono-substituted flavones, di-substituted flavones, and mixtures thereof; one or more isoflavones; coumarins selected from the group consisting of unsubstituted coumarins, mono-substituted coumarins, di-substituted coumarins, and mixtures thereof, chromones selected from the group consisting of unsubstituted chromones, mono-substituted chromones (including 3-formyl chromone), di-substituted chromones, and mixtures thereof; one or more dicoumarols; one or more chromanones; one or more chromanols; isomers (e.g., cis/trans isomers) thereof, and mixtures thereof. By the term “substituted” as used herein means flavonoids wherein one or more hydrogen atom of the flavonoid has been independently replaced with hydroxyl, C₁-C₈ alkyl, C₁-C₄ alkoxyl, O-glycoside, and the like or a mixture of these substituents.

The flavonoid compounds can be synthetic materials or obtained as extracts from natural sources (e.g., plants). The naturally sourced material can also further be derivatized (e.g., an ester or ether derivative prepared following extraction from a natural source). Flavonoid compounds useful herein are commercially available from a number of sources, e.g., Indofine Chemical Company, Inc. (Somerville, N.J.), Steraloids, Inc. (Wilton, N.H.), and Aldrich Chemical Company, Inc. (Milwaukee, Wis.). Preferred naturally sourced materials include kava root (standardised to give a kavalactone content of about 30% by wt and containing the full spectrum of lactones found in the kava plant) and green tea solids containing the full range of green tea polyphenols (i.e. catechins and epicatechins).—such materials may, optionally, be ingested as part of an oral composition.

Mixtures of flavonoid compounds may also be used.

Other suitable additives or skin actives are discussed in further detail in WO 97/39733, published Oct. 30, 1997, to Oblong et al., previously incorporated by reference in its entirety.

Optional Components: Topical Compositions

Compositions optionally comprise a pigment or mixture of pigments. The pigment used herein must be compatible with any acidic skin care active which may be present in the composition and have excellent overall colour stability. Suitable pigments for use herein can be inorganic and/or organic. Also included within the term pigment are materials having a low colour or lustre such as matte finishing agents, and also light scattering agents. Examples of suitable pigments are iron oxides, rutile titanium dioxide, anatase titanium dioxide, ferric oxide, ferrous oxide, chromium oxide, chromium hydroxide, manganese violet, acylglutamate iron oxides, ultramarine blue, D&C dyes, carmine, and mixtures thereof. Depending upon the type of make-up composition, e.g. foundation or blusher, a mixture of pigments will normally be used.

If the composition is a foundation, then the foundation composition can also include at least one matte finishing agent. The function of the matte finishing agent is to hide skin defects and reduce shine. Such cosmetically acceptable inorganic agents, i.e., those included in the CTFA Cosmetic Ingredient Dictionary, Third Ed., as silica, hydrated silica, silicone-treated silica beads, mica, talc, polyethylene, titanium dioxide, bentonite, hectorite, kaolin, chalk, diatomaceous earth, attapulgite zinc oxide and the like may be utilized.

An optional component of the topical compositions herein is a humectant or mixture of humectants, which can act as skin conditioners and are, therefore, to be considered as skin actives. The humectant or mixture of humectants herein is optionally present in an amount of from about 0.1% to about 30% preferably from about 1% to about 25%, and more preferably from about 1% to about 10% by weight of composition. Other conventional skin care product additives may also be included in the compositions of the present invention. For example, urea, guanidine and mixtures thereof may be used. Glycerine is a preferred humectant.

The topical compositions herein can additionally comprise an emollient. Emollients suitable for the compositions of the present invention include natural and synthetic oils selected from mineral, vegetable, and animal oils, fats and waxes, such as petrolatum, fatty acid esters, fatty alcohols, alkylene glycol and polyalkylene glycol ethers and esters, fatty acids and mixtures thereof.

Another optional component herein is one or more additional chelating agents, preferably in the range of from about 0.02% to about 0.10% by weight, based on the total weight of the composition. Preferably, the chelating agent is present in a concentration in the range of between about 0.03% and about 0.07% by weight, based on the total weight of the composition. Among the chelating agents that may be included in the composition is tetrasodium EDTA.

Another optional but preferred component of the topical composition is one or more preservatives. The preservative concentration in the composition, based on the total weight of that composition, is in the range of between about 0.05% and about 0.8%, preferably between about 0.1% and about 0.3%. Suitable preservatives for use herein include sodium benzoate and propyl paraben, and mixtures thereof.

Oral Compositions

Oral compositions are generally intended to induce satiety/promote nutrient malabsorbtion and thereby indirectly enhance thermogenesis and/or directly enhance thermogenesis to consume fat/calories and/or stimulate metabolic activity in general and lipolytic activity in particular.

Oral dosage forms are optional compositions for use in the present invention and these include the known forms for such administration, for example tablets, capsules, granules, syrups and aqueous or oil suspensions. Any carriers known in the art for oral application compositions may be used. For solid form preparations, such as, for example, powders, tablets, disbursable granules and capsules, a solid carrier may be one or more substances such as diluents, flavoring agents, solubilizers, lubricants, suspending agents, binders, tablet disintegrating agents, encapsulating materials and the like. Suitable carrier materials may include, for example, magnesium carbonate, calcium carbonate, sodium bicarbonate, magnesium stearate, calcium stearate, talc, lactose, sugar, pectin, dextrin, starch, tragacanth, cellulose derivatives, methyl cellulose, sodium carboxymethyl cellulose, a low-melting wax, cocoa butter, alginates, gelatin, polyvinyl pyrrolidone, polyethyl glycols, quaternary ammonium compounds and the like.

Tablets may be prepared from an active agent (nutrient absorption suppressant(s) and/or thermogenic agent(s)) or a mixture thereof (see below), with fillers, for example, calcium phosphate; disintegrating agents, for example, maize, starch; lubricating agents, for example, magnesium stearate; binders, for example, microcrystalline cellulose or polyvinylpyrrolidone and other optional ingredients known in the art to permit tableting the mixture by known methods. The tablets may, if desired, be coated using known methods and excipients which may include enteric coating using for example hydroxypropylmethylcellulose phthalate. The tablets may be formulated in a manner known to those skilled in the art so as to give a sustained release of a suitable active agent(s). Such tablets may, if desired, be provided with enteric coatings by known methods, for example by the use of cellulose acetate phthalate. Similarly, capsules, for example hard or soft gelatin capsules, containing the active agent(s) with or without added excipients, may be prepared by known methods and, if desired, provided with enteric coatings in a known manner. The contents of the capsule may be formulated using known methods so as to give sustained release of the active agent(s).

Other dosage forms for oral administration include, for example, aqueous suspensions containing an active agent(s) in an aqueous medium in the presence of a non-toxic suspending agent such as sodium carboxymethylcellulose, and oily suspensions containing the active agent(s) in a suitable vegetable oil, for example arachis oil. The active agent(s) may be formulated into granules with or without additional excipients.

The granules may be ingested directly by the patient or they may be added to a suitable liquid carrier (for example, water) before ingestion. The granules may contain disintegrants, e.g. an effervescent couple formed from an acid and a carbonate or bicarbonate salt to facilitate dispersion in the liquid medium.

Nutrient Absorption Suppressants: Oral Compositions

Active agents which act on the central nervous system (CNS) to suppress appetite and, therefore, suppress nutrient absorption may be used in the present oral compositions. One major subclass of CNS appetite suppressant drugs interacts with catecholaminergic receptors in the brainstem. These include controlled drugs such as amphetamine, phenmetrazine, and diethylproprion, and over-the-counter drugs such as phenylpropanolamine. Manizidol is another CNS active drug which, although not a catecholamine, activates the central nervous system. Oleic acid and salts and esters thereof are preferred nutrient absorption suppressants.

Other suitable active agents are drugs which promote malabsorption of nutrients through suppression of digestive enzymes. One agent in this category is Acarbose, a bacterial inhibitor of amylase and brushborder glycosidases. Another is tetrahydrolipostatin, a fungal inhibitor of lipases. These agents work by preventing digestion of carbohydrates and/or fats, thus creating an effective reduction in the number of calories absorbed, despite continued consumption.

Satiety inducing agents induce a feeling of satiety (suppress appetite) resulting in a net reduction in caloric intake following ingestion, shifting the balance of the body to enhanced lipolysis. Oleic acid and its esters are preferred satiety inducing agents.

Thermogenic Agents: Oral or Topical Compositions

Thermogenic agents, which act by promoting either metabolic activity in general or lypolytic activity in particular, may also be included in the present oral compositions. The catecholamine drugs discussed above have some thermogenic activity, in addition to their suppression of appetite. Thyroid hormone is also optionally used. The thermogenic agent may also include one or more of kola nut, N-acetyl-L-carnitine, cayenne extract, salicin, niacin or a derivative thereof (including niacinamide) or inositol hexanicotinate. N-acetyl-L-carnitine is useful in facilitating the transport of fat into mitochondria for their metabilization to generate energy. Cayenne extract stimulates the production of energy in the form of adenosine triphosphate (ATP) which, in turn, metabolizes more fat. Salicin, which is found naturally in the bark of the white willow, also has been implicated in the stimulation of thermogenesis. Niacin, also known as vitamin B-3, and its derivatives are known to induce thermogenesis and act to lower low density lipoprotein (LDL) cholesterol levels and elevate high density lipoprotein (HDL) cholesterol levels. It does so by reducing lipoprotein synthesis in the liver.

Agents which have a heating effect when applied on the skin, e.g., rubifacients, are also considered to be thermogenic agents.

Lipolytic agents are preferred thermogenic agents. A large number of active lipolytic agents may be used in the present compositions, such as asiatic acid; methylxanthines including caffeine, theophylline and aminophylline; nicotinic acid derivatives, such as α-tocopherol nicotinate or hexyl nicotinate; silicon; carnitine; coenzyme Q; escin; ruscogenin; draining, firming, lipolytic or veinotropic plant extracts; anti-glucose-uptake active agents; α-2-blocker compounds capable of blocking the α-2 receptors at the surface of adipocytes, such as ginkgo biloba; keratolytic agents, such as 5-octanoylsalicylic acid; salicylic acid; α-hydroxy acids such as lactic acid, malic acid, glycolic acid or tartaric acid or α-hydroxy acids from fruit, such as citric acid; polyethylene glycol fatty acid esters, glycerophosphatides, phosphatidylephosphates, egg yolk lecithin, oleic acid, stearic acid, palmitate, cholesterol, mono, di, and tri-glycerides, cholesterol ester, yolk lecithin containing 5 to 20% phosphatidic acid, linoleic acid, linolenic acid, lauric acid, phosphatidyl phosphate, glycerine, soy bean oil, sesame seed oil, and tromethan. Green tea solids also induce lipolysis by acting on adipocyte cells, thereby reducing fat mass of the body.

The following examples demonstrate the following for treatment of regional fat deposits including cellulite, et al (pain, e.g., and other conditions as already outlined in the case):

1. The device itself, and in combination with other devices

2. The device as a kit with topical compositions

3. Device, topical composition and oral composition as a kit or programme

4. At least 1 of the elements above with a monitoring function as part of a programme (% body fat monitoring, e.g.) or a business practice—home monitoring or at a spa, exercise club, etc.

As additional disclosure, the topical compositions include active agents for treating regional fat deposits including cellulite with the object of rebuilding the dermis (stimulate collagen, revasculaturize); anti-inflammatory action; anti-histamine action; lipolysis; hormonal therapy; and/or thermogenesis.

Without wishing to be bound by theory, it is believed that the action of the device (including the device combinations) achieves its objection by one or more of the following mechanisms of biostimulation; enhanced streaming; enhanced lymphatic drainage; promotion of tissue vascularization; cavitation; and/or increased blood flow (massage, heat, etc.)

### EXAMPLE 1

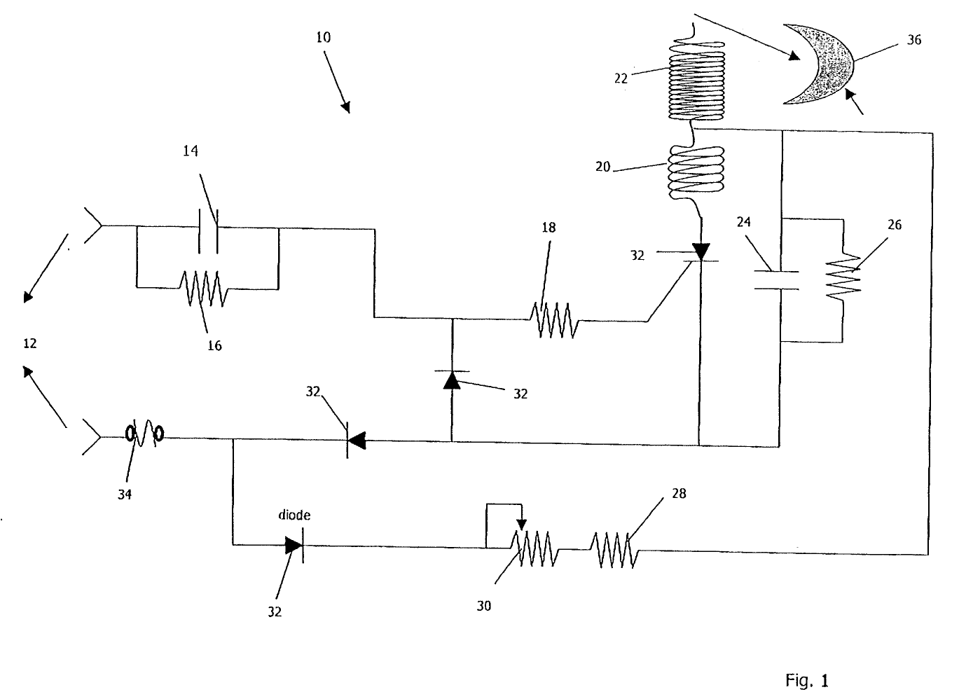

An electrostatic device (not shown) is prepared according to FIG. 1 of the drawings. The electrostatic device consists of a conventional circuit 10 ranged to effect the electrostatic application of a current from the outside of a glass bulb 36 to the skin, and also in the opposite direction. The circuit 10 includes an alternating electrical current source 12, at a voltage of 100 volts, in order to provide an electric current to the glass bulb 36. The circuit 10 includes a first capacitor 14 having a capacitance of 0.47 μF, a first resistor 16 with a resistance of 3.3 MΩ, a second resistor 18 having a resistance of 1.5 kΩ, a second capacitor 24 having a capacitance of 0.47 μF, a third resistor 26 having a resistance of 2 MΩ, a fourth resistor 28 having a resistance of 22 kΩ, a variable resistor 30 having a maximum resistance of 20 kΩ, and a fuse 34 to prevent overloading of the circuit 10. The circuit 10 also includes a plurality of diodes 32 disposed between the various components, to insure the correct flow of current through the circuit 10. The circuit 10 additionally includes a first coil 20, and a second coil 22 disposed in close proximity to the first coil 20, the second coil 22 having a relatively large number of windings. Both the first coil 20 and the second coil 22 are disposed within the glass bulb 36.

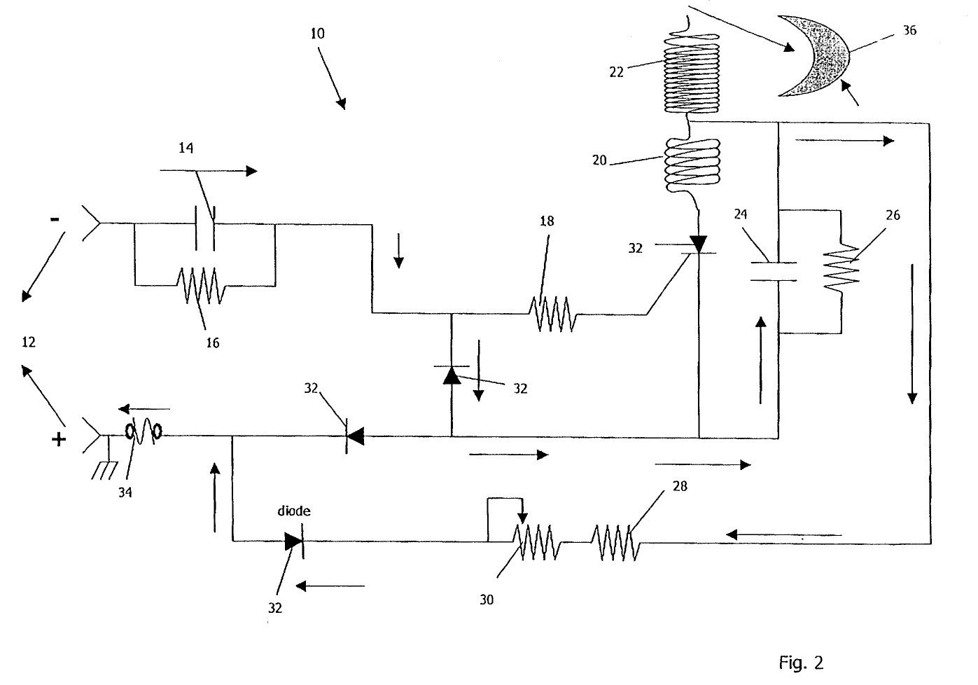

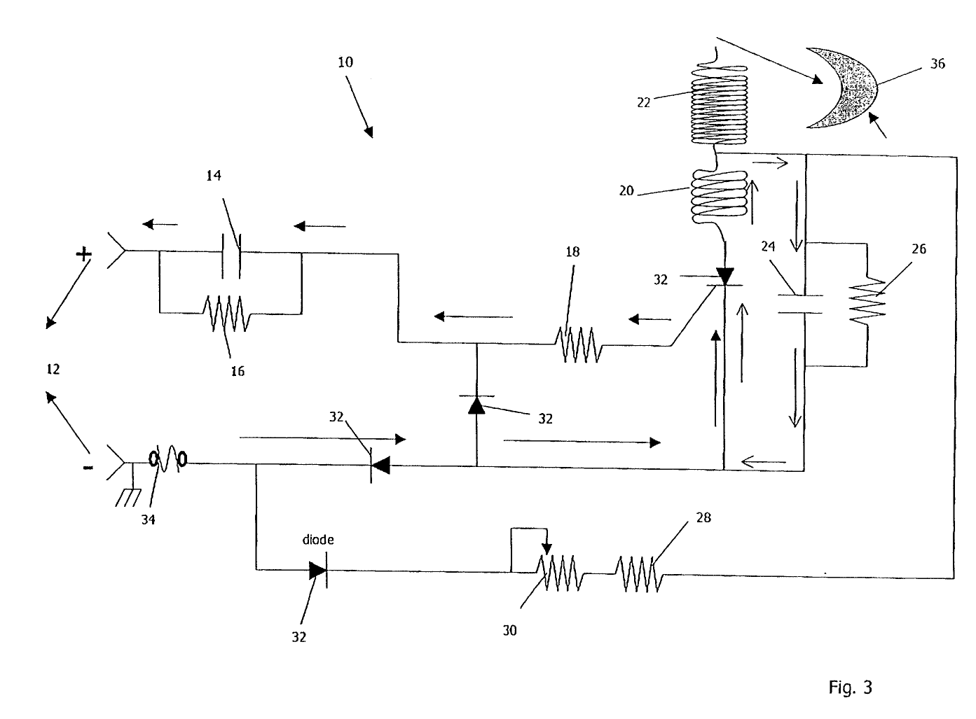

The device is fabricated inside a plastic housing, permanently insulating the electronic components away from the user. Both the 1st coil 20 and the 2nd coil 22 are recessed inside the vapor filled glass bulb 36, which diffuses electrical current as it flows from the 2nd coil 22 to the glass bulb 36 and vice versa, depending on the phase of the alternating electrical current source. As the 2nd capacitor 24 discharges through the first coil 22, the rapid growth and collapse of the electromagnetic field induces a current in the second coil, which acts as a transformer with a spark gap at the tip, discharging electrostatically through the glass bulb 36. FIG. 2 of the drawings shows the circuit 10 of FIG. 1, illustrating the steady state current flow for one half of the alternating current cycle. FIG. 3 illustrates the steady state current flow for the second half of the alternating current cycle. The glass bulb 36 measures 1/2 inch across.

The variable resistor 30 is set to induce maximum current, and the glass bulb 36 is placed in contact with an area of the thigh exhibiting signs of regional fat deposits including cellulite, treating the area for a period of 2 minutes per square inch of treatment area. Current jumps electrostatically from the outside of the glass bulb 36 to the skin, and also in the opposite direction. A shielded oscilliscope can be used to measure bi-directional electrostatic discharge in the range of 2,000-3,000 volts with this device, as is typically for electrostatics. Trans epidermal water loss (TEWL) is measured before and after treatment using a commercial TEWL meter with recording software, such as DermaLab® System with TEWL probe available from cyberDERM, Inc., Media, Pa., USA (www.cyberderm-inc.com). The following results are obtained.

| Time | TEWL (gms/m²/hr) |
|---|---|
| Baseline | 8.2 |
| 1 minute after treatment | 19.1 |
| 30 minutes after treatment | 14.3 |
| 4 hours after treatment | 11.1 |

The increased TEWL indicates effective flow of electrical current across the stratum corneum and into the tissues, beneficial in stimulating the tissues for the repair of regional fat deposits including cellulite. For about 1 hour after treatment, the treated area is reddened, indicating increased bloodflow, also beneficial for the treatment of regional fat deposits including cellulite.

### EXAMPLE 2

A massaging device is used in combination with the electrostatic stimulating device from Example 1. A massage is given to the thigh region for 15 minutes using a commercially available anti-cellulite pinching roller vacuum device, the Cellesse SenseActive HP5231 device manufactured by Philips. After massage, the electrostatic device of Example 1 is used to treat the skin as described, for the same treatment duration described in Example 1. The massage physically breaks down parts of the tissue that contribute to regional fat deposits, and the enhanced circulation provided by the electrostatic device flushes the products from the site for clearance by the body. Combining the two steps into a single device is anticipated.

### EXAMPLE 3

An electret is prepared which is a polypropylene substrate having a basis weight of about 88 grams per square meter and having a surface charge of at least about 7,000 volts. The process for preparing this electret is detailed for example in U.S. Pat. No. 4,142,521. One such commercially available electret is the Pain T.E.M. Therapeutic Electro Membrane available by ordering at the web site http://www.paintem.com. Surface charge is measured by an electrostatic voltmeter, for example the Trek Model 523 hand-held electrostatic voltmeter, measured with the electrode in contact with the substrate. The electrostatic voltmeter is available from Trek, Inc., at http://www.trekinc.com/523sp.htm. The electret is held on the thigh where there is a visible appearance of cellulite, and a sleeve is placed on the thigh, which compresses the thigh and holds the electret in place. An exemplary sleeve is the Donjoy Thigh Sleeve available from dj Orthopedics, LLC, Vista, Calif., USA. The electret is worn for 6 hours and discarded. A new electret is used daily, and treatments are applied daily for 2 months to visibly reduce the signs of regional fat deposits including cellulite.

### EXAMPLE 4

A kit is prepared comprising a topical therapeutic cream and a device, and is used to treat regional fat deposits including cellulite. A skin cream is prepared by combining and mixing the following ingredients using conventional technology.

| Ingredient | % Weight |
|---|---|
| Glycerine | 6.933 |
| Niacinamide | 14.00 |
| Theophylline | 1.500 |
| Caffeine | 0.500 |
| Permethyl 101A¹ | 3.000 |
| Sepigel² | 2.500 |
| Q2-1403³ | 2.000 |
| Isopropyl Isostearate | 1.330 |
| Arlatone 2121⁴ | 1.000 |
| Cetyl Alcohol CO-1695 | 0.720 |
| SEFA Cottonate⁵ | 0.670 |
| Tocopherol Acetate | 0.500 |
| Panthenol | 0.500 |
| Adol 62⁶ | 0.480 |
| Kobo Titanium Dioxide | 0.400 |
| Sodium Hydroxide 50% Aqueous | 0.0150 |
| Fiery 5⁷ | 0.150 |
| Disodium EDTA | 0.100 |
| Glydant Plus⁸ | 0.100 |
| Myrj 59⁹ | 0.100 |
| Emersol 132¹⁰ | 0.100 |
| Color | 0.00165 |
| Purified Water | q.s. to 100 |

¹Isohexadecane, Presperse Inc., South Plainfield, NJ;
²Polyacrylamide(and)C13-14 Isoparaffin(and)Laureth-7, Seppic Corporation, Fairfield, NJ;
³dimethicone(and)dimethiconol, Dow Corning Corp., Midland, MI;
⁴Sorbitan Monostearate and Sucrococoate, ICI Americas Inc., Wilmington, DE;
⁵Sucrose ester of fatty acid, Procter and Gamble, Cincinnati, OH;
⁶Stearyl alcohol, Procter and Gamble, Cincinnati, OH;
⁷Fiery, Procter and Gamble, Cincinnati, OH;
⁸DMDM Hydantoin (and) Iodopropynyl Butylcarbamate, Lonza Inc., Fairlawn, NJ;
⁹PEG-100 Stearate, ICI Americas Inc., Wilmington, DE;
¹⁰Stearic acid, Henkel Corp., Kankakee, IL.

The device of Example 1 is used to treat the outer thigh which exhibits regional fat deposits dimples, treating for a period of 2 minutes per square inch of treatment area. The topical composition is applied to the treated area immediately after treatment with the electrostatic device, which treatment facilitates absorption of the cream through the skin. Treatment is continued daily until regional fat deposits are diminished.

### EXAMPLE 5

A disposable wrap is prepared which provides heat and electrostatic energy simultaneously. A disposable thermal wrap is prepared which comprises individual heat cells of oxygen-activated exothermic disks contained between two continuous nonwoven layers. One-inch diameter and 0.5 inch thick heat disks are prepared and compacted in the manner disclosed in U.S. Pat. No. 6,020,040. Twenty-four disks are prepared and sealed in pockets between a layer of impermeable film and a layer of film having an oxygen permeability of 3 cc 02/min./5 cm2 (at 21° C., 1 ATM), spaced 3 inches apart center-to-center in a hexagonal packing array. The electret of example 3 is placed on the inside of the heat-generating wrap (the skin contacting side) and the combination wrap is wrapped around the thigh to provide a combination of electrostatic potential energy and heat to the skin simultaneously.

### EXAMPLE 6

A kit is prepared comprising a topical therapeutic cream and a device, and is used to treat regional fat deposits including cellulite. An emulsion is prepared by first creating the water phase and then creating the oil phase. After both phases are created, they are mixed together and retinyl palmitate is added. The water phase is made by first weighing deionized water into a beaker and, with mixing at high speed, slowly adding carboxy polymer. EDTA and ascorbic acid are then added to the mixture and mixing is continued until well-dissolved, about 40 minutes. The water phase is then heated to 80° C., at which time propylene glycol is added. To make the oil phase, all ingredients of the oil phase are weighed and added together in a separate beaker, heating to 80° C. with mixing until homogeneous. The oil phase is then slowly poured into the water phase with mixing. Sodium hydroxide is added at 80° C. in order to adjust the pH of the emulsion. After mixing for ten minutes, the emulsion is cooled to 45° C. Retinyl palmitate is then added to the emulsion and the emulsion is mixed until homogeneous. The procedure is carried out under yellow light and under a nitrogen blanket so as to minimize exposure to oxygen.

| | Content |
|---|---|
| Ingredient | (% W/W) |
| Carboxyvinyl polymer | 0.300 |
| Propylene glycol | 5.00 |
| Methylparaben | 0.15 |
| Ascorbic Acid | 0.10 |
| Glyceryl monostearate | 5.00 |
| Cetanol | 1.00 |
| Stearyl alcohol | 0.50 |
| White Petrolatum | 1.50 |
| BHT | 0.05 |
| Propylparabsn | 0.10 |
| Butylparaben | 0.05 |
| Cetyl palmitate | 1.00 |
| C12-C15 Alkyl Benzoate | 4.00 |
| Benzyl alcohol | 0.30 |
| Ethyl alcohol | 4.00 |
| Disodium EDTA | 0.05 |
| Retinyl palmitate | 0.30 |
| Sodium Hydroxide (10%) | to adjust pH to 8.0 |
| Water | QS |

The electret of example 3 is used to treat the outer thigh which exhibits cellulite dimples. Each day, after the electret treatment is completed, the topical composition is applied to the treated area immediately after treatment, which treatment facilitates absorption of the cream through the skin. Treatment is continued daily until signs of regional fat deposits are diminished.

### EXAMPLE 7

A kit is prepared comprising a topical therapeutic cream, a device and an oral composition, and is used to treat regional fat deposits including cellulite.

An oral composition is prepared as follows. A single packet of a dry instant beverage mix is blended with 4.0 grams of ethyl oleate. The dry instant beverage mix is sold by Nestle® Carnation® as French Vanilla Flavored Instant Breakfast™ Nutritional Energy Drink (or similar) and comprises about 36.3 grams of dry instant mix. The dry mix contains these ingredients; nonfat dry milk, maltodextrin, sugar, cellulose gum, natural and artificial vanilla flavor, dicalcium phosphate, magnesium hydrochloride, sodium ascorbate, ferric orthophosphate, vitamin E acetate, niacinamide, copper gluconate, zinc oxide, calcium pantothenate, manganese sulfate, vitamin A palmitate, pyridoxine hydrochloride, thiamin mononitrate, folic acid, biotin, phylloquinone, vitamin B12. The ethyl oleate is stirred into the dry mixture. An oral composition beverage is prepared from the resulting mixture by stirring into 8 fluid ounces of skim milk. The beverage (oral composition) is consumed as breakfast (in place of other food for breakfast) as part of an extended program to reduce the appearance of regional fat deposits including cellulite. The beverage induces a feeling of satiety resulting in a net reduction of calorie intake over the course of the day the beverage is consumed, contributing to a reduction in regional fat deposits including cellulite, as part of the program. The electret sleeve of Example 6 is applied as a wrap to the leg and worn each day as part of the program. The topical therapeutic cream of Example 6 is applied topically to the thighs once per day after the electret has been removed, as part of the program. The components of the kit work synergistically to reduce caloric intake, necessitating lipolysis and preferentially lipolysis from adipocytes located in the thigh region due to the device and cream.

### EXAMPLE 8

An area of a patient's thigh which exhibits signs of regional fat deposits such as cellulite is treated with electrical energy and ultrasound energy. Integration of the energies into a single device is preferred, although each may be separately provided.

An electrical device is prepared according to example 1. The device applies alternating electrical current to tissues from an electrostatic hand-held device of about 2,000 volts, alternating in polarity. The adjustable resistor is set to apply maximum electrostatic potential through the skin contact bulb (about one half inch diameter), and an isolated area of a woman's outer thigh is treated with the device. The area treated measures approximately 20 square inches. The site is treated by moving the device slowly in a circular motion over the skin for 22 minutes. Laser doppler blood flow of the electrostatically treated area prior to and 15 minutes after electrostatic treatment confirms improved circulation to the area. 15 minutes after electrostatic treatment, the same area of the skin is subjected to ultrasound treatment which is provided using a commercially available ultrasound apparatus (Mettler Sonicator 730, available from Mettler Electronics Corporation (http://www.mettlerelec.com/ultrasnd.html)). Ultrasound energy at 3 MHz is applied through a hand-held transducer which has a 5 cm² skin contact area. A mediating gel is spread on the outer thighs of the subject prior to treatment. Ultrasound energy is continuously applied at a power density of 0.2 Watts per square centimeter (W/cm²) to an area of the thigh measuring about 300 cm² for a period of 15 minutes. The ultrasound probe is continuously moved in a slow, circular motion within the treatment area. The treatment is repeated as often as necessary until the signs of regional fat deposits, and in particular cellulite, are reduced.

### EXAMPLE 9

Electromagnetic radiation is used in combination with the electrostatic stimulating device from Example 1. A light patch array is fabricated using Gallium Arsenide Phosphide on Gallium Phosphide red light emitting diodes (LEDs) which illuminate maximally at about 635 nm. Spectral analysis is verified with a spectrometer, for example Ocean Optics SD2000 High Sensitivity Fiber Optic Spectrometer with OOiBase32 PC software, from Ocean Optics, Inc. Agilent Technologies HLMP-1340 T-1 diodes are used. Each of the diodes measures approximately 3 mm diameter with transparent lenses and a 45 degree viewing angle. An individual diode delivers about 0.10 milliwatts (mW) optical power at 1.85 volts; 0.16 mW optical power at 1.95 volts; and 0.32 mW optical power at 2.14 volts, drawing 8.0, 15.1 and 30.3 milliamps (mA) current, respectively, at the specified voltages. Optical power is measured with a multifunctional optical power meter, for example an Oriel OPM Model 70310 with enhanced UV Silicone Detector, a 1 cm square array, and the LED positioned as close as possible to the detector (8 mm). The LEDs are connected in parallel by soldering to a standard rigid printed circuit board with 0.1 inch (0.254 cm) grid using a diode density of 25 two-pin diodes per square inch (6.45 cm²) (i.e., 50% of PC board capacity). The PC board measures 6 inches (15.24 cm) by 4 inches (10.16 cm), with 438 diodes covering an inner 5 inch (12.7 cm) by 3.5 inch (8.89 cm) rectangular area of the board. Two six-cell rechargeable NiMH batteries are connected in series to deliver power through a DC-DC switching converter to reduce voltage to approximately 2.0 volts, and the voltage is trimmed using an adjustment circuit and potentiometer to deliver 1.95 volts to the array, measured across each diode. The array has an optical power of about 0.60 mW/cm². A small, battery powered fan is affixed to the back of the array to remove excess heat generated during use. The array is affixed to an elastic neoprene sleeve (5 mm thick) measuring about 25 inches (63.5 cm) long by 8.5 inches (21.59 cm) wide that has two, 2-inch (5.08 cm) wide elastic straps that extend another 10 inches (25.4 cm) in length. The straps are attachable and detachable to the bulk of the sleeve by a hook and loop type fastening system, to affix the sleeve to the thigh while concurrently allowing therapeutic compression to be applied. A rectangular hole is cut in the center of the neoprene sleeve, and straps located at its edge to allow the light patch array to sit within the sleeve. Wires connect the array to the power supply. The power supply and battery are contained in a pouch with a hook to attach to the belt or waistband of the user, so the sleeve can be worn while the user is active. The sleeve is attached to the thigh of a user to treat regional fat deposits including cellulite, the power supply switched on, and the user resumes normal activity for a period of between 0.5 and 2 hours, applying about 1 to 4 Joules/cm² (J/cm²). After this period, the sleeve is rotated or moved to apply energy to a different site, moved to the other leg, or removed. By applying energy to different sites, the entire thigh is treated with light and therapeutic compression. After about 4 hours of continuous use, the batteries are recharged to prepare them for another cycle. After the application of electromagnetic radiation, the electrostatic device of Example 1 is used to treat the skin as described, for the same treatment duration described in Example 1.

### EXAMPLE 10

One inch diameter neodymium disc magnets having a field strength of 1,000 Gauss are selected (available commercially from many manufacturers and distributors of therapeutic magnets, for example ForceField at www.wondermagnet.com or telephone (970)484-7257 USA). Twenty magnets are stitched into the inside of Lycra™ bicycle shorts around each thigh and additional 20 magnets along the buttocks area. The magnets are placed at 2 inch intervals in a square pattern (center to center distance) with field direction alternating N and S in adjacent magnets. A heavy cardboard support is placed into the center of the shorts to separate the magnets when not in use. The electret of example 3 is wrapped around the thigh on the inside of the bicycle shorts in order to provide a combination of electrostatic potential energy and magnetic energy to the skin simultaneously.

### EXAMPLE 11

A capsule is prepared. 5.0 gm pharmaceutical grade caffeine are dry blended with 10.0 gm dried kava root (standardized to provide a kavalactone content of about 30% by wt and containing the full spectrum of lactones found in the kava plant) and 20.0 gm dried green tea solids (prepared by freeze drying the boiled water extract of green tea leaves and containing the full range of green tea polyphenols, i.e., catechins and epicatechins). The dried mixture is filled into gelatin capsules, 900 mg per gelcap.

A microemulsion is prepared. A mixture of 11 gm retinyl palmitate is prepared with 5 gm tocopherol, 1 gm ergocalciferol, 18 gm soybean oil, 10 gm tricapric acid glyceride (MCT), 9 gm decaglycerol laurate (HLB 15.5), 21 gm decaglycerol myristate (HLB 14) and 8 gm sucrose stearate (HLB 19). The components are heated to 65 degrees C. until melted and homogeneous, with stirring. After returning to room temperature, 100 gm of 85% propylene glycol aqueous solution is added and stirred until a homogeneous cream results. The cream is diluted 50-fold with purified water to form a microemulsion having a particle size of about 130 nm.

The gelcaps are administered orally to a woman exhibiting signs of regional fat deposits such as cellulite and obesity throughout the day for a period of 12 weeks, three tablets per day taken 45 minutes before each meal. Twice daily, about 3 grams of the microemulsion is applied to her thigh and buttocks region. Once per day, the electret sleeve of Example 3 is worn on at least one thigh for at least 2 hours per location.

Body weight, body fat content, thigh circumference measurements, and a regional fat deposit visual (expert) grade are taken prior to the program commencement and at its conclusion, demonstrating an improvement in weight and regional fat deposit signs. It will be appreciated that the program may further comprise using a source of at least one alternative energy form selected from the group comprising ultrasound, electrotherapy, active massage, static magnets, heat and combinations thereof.

### EXAMPLE 12

A capsule is prepared. 5.0 g pharmaceutical grade caffeine are dry blended with 10.0 g dried kava root (standardized to provide a kavalactone content of about 30% by wt and containing the full spectrum of lactones found in the kava plant) and 20.0 g dried green tea solids (prepared by freeze drying the boiled water extract of green tea leaves and containing the full range of green tea polyphenols, i.e., catechins and epicatechins). The dried mixture is filled into gelatin capsules, 900 mg per gelcap.

A microemulsion is prepared. A mixture of 11 g retinyl palmitate is prepared with 5 g tocopherol, 1 g ergocalciferol, 18 g soybean oil, 10 g tricapric acid glyceride (MCT), 9 g decaglycerol laurate (HLB 15.5), 21 g decaglycerol myristate (HLB 14) and 8 g sucrose stearate (HLB 19). The components are heated to 65 degrees C. until melted and homogeneous, with stirring. After returning to room temperature, 100 g of 85% propylene glycol aqueous solution is added and stirred until a homogeneous cream results. The cream is diluted 50-fold with purified water to form a microemulsion having a particle size of about 130 nm.

The gelcaps are administered orally to a woman exhibiting signs of regional fat deposits including cellulite and obesity throughout the day for a period of 12 weeks, three tablets per day taken 45 minutes before each meal. Twice daily, about 3 grams of the microemulsion is applied to her thigh and buttocks region. Once per day, the electret and sleeve arrangement of Example 3 is worn on at least one thigh for at least 4 hours per location.

The above described arrangements are merely illustrative of the principles of the present invention. Other modifications and adaptations may occur to those skilled in the art, without departing from the spirit and scope of the present invention.

What is claimed is:

1. A device for treating a selected area of cosmetic skin conditions such as regional fat deposits, the device comprising a storage medium from which, in use, a current is electrostatically discharged between the selected area and the device.

2. A device according to claim 1 wherein the current is in the range of between 1 microamps and 1000 microamps.

3. A device according to claim 1 wherein the storage medium has a potential thereon of between 0.5 kilovolts and 20 kilovolts.

4. A device according to claim 1 wherein the storage medium is an electret.

5. A device according to claim 1 wherein the storage medium is the form of a diffuser.

6. A device according to claim 5 wherein the diffuser is an air filled glass bulb.

7. A device according to claim 5 wherein the potential is applied to the diffuser from an induction coil.

8. A device according to claim 5 wherein the potential applied to the diffuser is an alternating potential.

9. A device according to claim 1 further comprising a source of at least one alternative energy form selected from the group comprising light, ultrasound, active massage, heat, compression, static magnets and combinations thereof, wherein the, or each, alternative energy form is applied at or adjacent the selected area.

10. A device according to claim 9 wherein the alternative energy form comprises ultrasound.

11. A device according to claim 10 wherein the ultrasound source comprises an ultrasonic generator for generating pressure waves having a frequency of at least 16 kHz.

12. A device according to claim 11 wherein the device includes an ultrasonic applicator for applying the ultrasonic output of said ultrasonic generator to the selected area.

13. A device according to claim 1 wherein the alternative energy form comprises active massage.

14. A device according to claim 13 wherein the device includes one or more mechanical massaging elements for application to the treatment area.

15. A device according to claims 1 wherein the alternative energy form comprises a static magnet.

16. A device according to claim 15 wherein the device includes a series of juxtaposed static magnets for application to the treatment area.

17. A method of treating a selected area of a cosmetic skin condition such as regional fat deposits by the electrostatic application, from a storage medium, of a current between the storage medium and the selected area.

18. A method according to claim 17 comprising the steps of;
(1) exposing the selected area to the current; and
(2) exposing the selected area to a source of at least one alternative energy form selected from the group comprising light, ultrasound, active massage, heat, compression, static magnets and combinations thereof.

19. A method according to claim 18 wherein the selected area is simultaneously exposed to the current and to the, or each, alternative energy form source.

20. A method according to claim 18 wherein the selected area is sequentially exposed, in either order, to the current and to the, or each, alternative energy form source.

21. A method according to claim 18 wherein the method includes applying a topical composition on or adjacent the selected area before, during, or after exposing the treatment area to the current and/or the, or each, alternative energy form.

22. A method according to claim 18 wherein the method includes administering an oral composition before, during or after exposing the selected area to the current and/or the, or each, alternative energy form.

23. A kit for treating a selected area of a cosmetic skin condition such as regional fat deposits including;
(1) a device comprising an electrostatic storage medium from which, in use, a current is electrostatically discharged between the device and selected area; and
(2) a topical composition adapted to be applied on or adjacent the selected area.

24. A kit according to claim 23 further including a device comprising at least one alternative energy form selected from the group comprising light, ultrasound, active massage, heat, compression, static magnets and combinations thereof, wherein the, or each, alternative energy form is applied at or adjacent the selected area.

25. A kit according to claim 23 wherein the current is in the range of between 1 microamp and 1000 microamps.

26. A kit according to claim 23 wherein the storage medium has a potential thereon of between 0.5 kilovolts and 20 kilovolts.

27. A kit according to claim 23 wherein the storage medium is an electret.

28. A kit according to claim 23 wherein the storage medium is in the form of a diffuser.

29. A kit according to claim 28 wherein the diffuser is an air filled glass bulb.

30. A kit according to claim 28 wherein the potential is applied to the diffuser from an induction coil.

31. A kit according to claim 23 further including an oral composition.

32. A kit according to claim 23 wherein the topical composition is selected from the group consisting of theophylline, aminophylline, niacinamide, retinyl palmitate and mixtures thereof.

33. A kit according to claim 23 wherein the oral composition includes caffeine, soy extracts, soy isolates including soy isoflavones.
